(19) United States
(12) Patent Application Publication
(10) Pub. No.: US 2026/0325295 A1
(43) Pub. Date: Oct. 22, 2026

(54) Devices and Methods for Loading Collapsible Prosthetic Heart Valve

(71) Applicant: St. Jude Medical, Cardiology Division, Inc., St. Paul, MN (US)

(72) Inventors: Alexander Barrette, Maplewood, MN (US); William Minion, Vadnais Heights, MN (US)

(73) Assignee: St. Jude Medical, Cardiology Division, Inc., St. Paul, MN (US)

(21) Appl. No.: 19/452,524

(22) Filed: Jan. 19, 2026

Related U.S. Application Data
(60) Provisional application No. 63/792,654, filed on Apr. 22, 2025.

(51) Int. Cl. A61F 2/24; A61F 2/95

(52) U.S. Cl. CPC A61F 2/2436; A61F 2/2418; A61F 2/95

(57) ABSTRACT

A method of loading a prosthetic heart valve into a delivery device may include advancing the prosthesis through a tapered compression member until a valve retainer protrudes from the compression member. Then, a tip of the delivery device may be advanced through the prosthesis until a retaining element of the delivery device is positioned adjacent to the retainer. The retainer may be positioned within a recess in the retaining element. A locking ring may be advanced from a first position on the delivery device to a second position in which the locking ring overlies either (i) the retainer or (ii) a portion of the stent adjacent to the first retainer, so that the locking ring locks the first retainer within the first recess. A distal sheath of the delivery device may be moved to cover the prosthesis.

### CROSS-REFERENCE TO RELATED APPLICATIONS

This application claims priority to the filing date of U.S. Provisional Patent Application No. 63/792,654, filed Apr. 22, 2025, the disclosure of which is hereby incorporated by reference herein.

### BACKGROUND

The present disclosure relates to prosthetic heart valve implantation and, more particularly, to assemblies and methods for loading a self-expanding collapsible heart valve into a delivery device.

Prosthetic heart valves may be formed from biological materials such as harvested bovine valves or pericardial tissue. Such valves are typically fitted within a stent or frame, which may be inserted into the heart at the annulus of the compromised native valve to replace the native valve. To perform such insertion procedure using a minimally invasive technique, it is typically necessary to compress the stent to a reduced diameter for loading into the delivery device.

In the case of valves formed from biological materials, the stented valve is preferably preserved in the open condition for storage as compression of the valve material for extended periods may compromise the integrity of the biological valve. In such instances, it may therefore be necessary to crimp the valve, or otherwise reduce its diameter for loading in the delivery device, in the operating arena.

Although crimping devices are known, there are still improvements that can be made to achieve more effective loading of a collapsible prosthetic heart valve on to a delivery device.

### SUMMARY OF THE DISCLOSURE

According to one aspect of the disclosure, a method of loading a self-expanding prosthetic heart valve into a delivery device includes advancing the prosthetic heart valve through a first end of compression member until a first retainer of the prosthetic heart valve protrudes from an open second end of the compression member, the compression member tapering from a larger diameter at the first end to a smaller diameter at the second end, the first retainer being formed at an end of a stent of the prosthetic heart valve. While the first retainer protrudes from the open second end of the compression member, a tip of the delivery device is advanced through the stent of the prosthetic heart valve until a retaining element of the delivery device is positioned adjacent to the first retainer. While the retaining element of the delivery device is positioned adjacent to the first retainer, the first retainer is positioned within a first recess formed in the retaining element. While the first retainer is positioned within the first recess, a locking ring is advanced from a first position on the delivery device in a distal direction toward the tip of the delivery device to a second position in which the locking ring overlies either (i) the first retainer or (ii) a portion of the stent adjacent to the first retainer, so that the locking ring locks the first retainer within the first recess. A distal sheath of the delivery device is moved from a proximal open position to a distal closed position, whereby moving the distal sheath results in the prosthetic heart valve being maintained in a collapsed condition within a compartment defined between the tip and the retaining element. The locking ring may be transparent, and the method may include visually confirming that the first retainer is properly positioned within the first recess while the locking ring overlies either (i) the first retainer or (ii) the portion of the stent adjacent to the first retainer. Advancing the locking ring from the first position to the second position may include coupling the locking ring to the second end of the compression member. The locking ring may include an annular rim, and coupling the locking ring to the second end of the compression member may include achieving a friction fit, a snap fit, or an interference fit between the annular rim and the second end of the compression member. Prior to advancing the tip of the delivery device through the stent of the prosthetic heart valve, a constricting member may be positioned over the delivery device, so that an enlarged head at an end of the constricting member faces toward the tip of the delivery device. The constricting member may be advanced until the locking ring is received within the enlarged head of the constricting member while the locking ring overlies either (i) the first retainer or (ii) the portion of the stent adjacent to the first retainer. The constricting member and/or the compression member may be transparent. The step of advancing the prosthetic heart valve through the first end of compression member may be performed after the prosthetic heart valve is positioned, while in an expanded condition, on a support member. The step of advancing the prosthetic heart valve through the first end of the compression member may include advancing the support member toward the compression member until the support member contacts first end of the compression member, and the method may further include reversibly coupling the support member to the compression member.

According to another aspect of the disclosure, a system includes a self-expanding prosthetic heart valve including a stent, a valve assembly supported by the stent, and a first retainer at one end of the stent, the prosthetic heart valve having an expanded condition and a collapsed condition. The system includes a delivery device including a tip, a retaining element having a first recess sized and shaped to receive the first retainer therein, a compartment at least partially defined between the tip and the retaining element and adapted to receive the prosthetic heart valve, and a distal sheath movable between a distal closed position fully covering the compartment and a proximal open position uncovering the compartment. The distal sheath is configured to maintain the prosthetic heart valve in the collapsed condition in the compartment when the distal sheath is in the distal closed position. A compression member has a first open end with a first diameter, a second open end with a second diameter less than the first diameter, and a wall decreasing in diameter from the first open end to the second open end, the wall defining an open space adapted to receive the prosthetic heart valve. The compression member is configured to radially compress the prosthetic heart valve as the prosthetic heart valve advances through the open space from the first open end toward the second open end. A locking ring having has an inner diameter corresponding to an outer diameter of the retaining element, the locking ring configured to slide over the delivery device and to overlie either (i) the first retainer or (ii) a portion of the stent adjacent to the first retainer while the first retainer is received within the first recess so that the locking ring locks the first retainer within the first recess. The prosthetic heart valve may include second and third retainers at the one end of the stent. The retaining element may have a second recess sized and shaped to receive the second retainer therein, and a third recess sized and shaped to receive the third retainer therein. The locking ring may be transparent. The locking ring may be configured to reversibly couple to the second open end of the compression member. The locking ring may include an annular rim configured to achieve a friction fit, a snap fit, or an interference fit with the second open end of the compression member. A constricting member may have an enlarged head at an end of the constricting member, the constricting member configured to be received over the delivery device with the enlarged head facing toward the tip of the delivery device. The enlarged head of the constricting member may have an interior diameter sized to receive the locking ring. The constricting member and /or the compression member may be transparent.

### BRIEF DESCRIPTION OF THE DRAWINGS

FIG. 1 is a perspective view of a distal portion of a delivery device.

FIG. 2 is a perspective view of a proximal portion of the delivery device of FIG. 1.

FIG. 3 is an enlarged side view of a retaining element of the delivery device shown in FIG. 1 and 2.

FIG. 4 is a perspective view of a self-expanding prosthetic heart valve.

FIG. 5 is a perspective view of a compression member.

FIG. 6A is a perspective view of a support member.

FIG. 6B is a side elevational view of the support member of FIG. 6A.

FIG. 6C is a cross-sectional view of the support member of FIG. 6A, taken along section line A-A of FIG. 6B.

FIG. 7 is a perspective view of a spacer.

FIG. 8 is a longitudinal cross-sectional view of a constricting member.

FIG. 9 is an enlarged longitudinal cross-sectional view of an end section of the constricting member of FIG. 8.

FIG. 10 is a longitudinal cross-sectional view of a loading assembly, including the compression member of FIG. 5, the support member of FIG. 6A, and the constricting member of FIG. 8.

FIGS. 11-20 illustrate exemplary steps of a method for loading a prosthetic heart valve into a delivery device using the loading assembly of FIG. 10.

FIG. 21 shows a loading assembly according to another aspect of the disclosure, including a base member, a compression member, a locking ring, and a constricting member.

FIG. 22 shows a stent of a prosthetic heart valve coupled to a delivery device, with the locking ring of FIG. 21 on the delivery device, and the compression member of FIG. 21 compressing the stent.

FIG. 23 shows the components of FIG. 22 after the locking ring has been engaged with an end of the compression member.

### DETAILED DESCRIPTION OF THE DISCLOSURE

Embodiments of the presently disclosed loading assemblies are described herein in detail with reference to the drawing figures, wherein like reference numerals identify similar or identical elements. In the drawings and in the description which follows, the term “proximal” refers to the end of the catheter assembly, or portion thereof, which is closest to the operator during intended use, and to the end of the loading assembly which is closest to the proximal end of the catheter assembly when the loading assembly is assembled on the catheter assembly during a valve loading procedure. The term "distal" refers to the end of the catheter assembly, or portion thereof, which is farthest from the operator in intended use, and to the end of the loading assembly which is closest to the distal end of the catheter assembly when the loading assembly is assembled on the catheter assembly during a valve loading procedure.

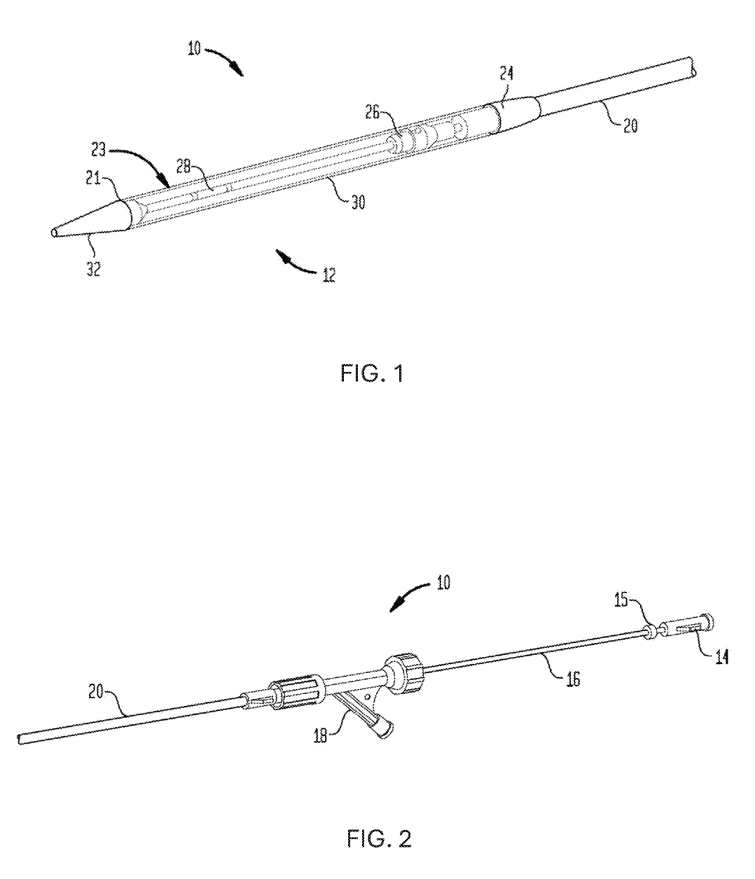

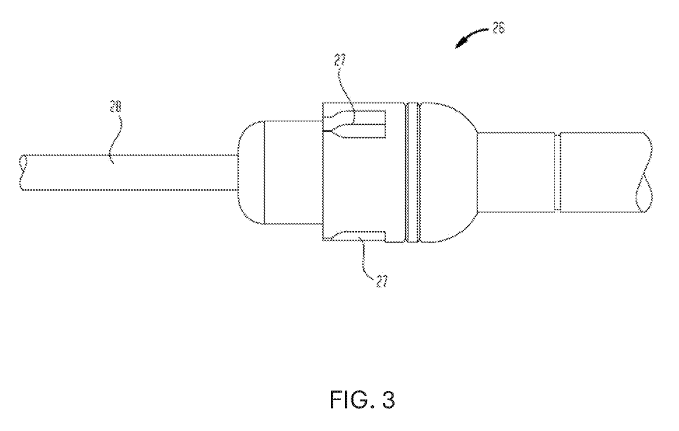

The present disclosure relates to assemblies and methods for loading a self-expanding stent or frame, or a collapsible prosthetic heart valve, into a minimally invasive delivery device. An exemplary minimally invasive delivery device 10 is illustrated in FIG. 1 and 2.

As shown in FIGS. 1 and 2, an exemplary delivery device 10 for transfemoral delivery of a collapsible prosthetic aortic valve (or other types of self-expanding collapsible stents, frames, or other prosthetic heart valves) includes a catheter assembly 12 for delivering the prosthetic heart valve to, and deploying the heart valve at, a target location. The catheter assembly 12 may include a compartment 23 defined between an atraumatic tip 32 of the delivery device 10 and a retaining element 26. A support shaft 28 may be connected between tip 32 and retaining element 26 and may define the length of compartment 23. A distal sheath 30 may be slidably arranged relative to the compartment 23 so that, in a distalmost or closed position in which the distal end 21 of the distal sheath 30 abuts atraumatic tip 32, the distal sheath 30 covers the prosthetic heart valve and retains it in a collapsed condition for delivery to the target site, and in a proximal or open position in which the distal end 21 of the distal sheath 30 is spaced from the atraumatic tip 32, the distal sheath 30 uncovers the prosthetic heart valve for deployment at the target site.

An inner tube 16 having a lumen therethrough may extend from a hub 14 at or near its proximal end to a distal end which may be connected to retaining element 26. Optionally, the distal end of inner tube 16 may extend through retaining element 26 and support shaft 28 for connection to atraumatic tip 32. In either arrangement, the distal end of inner tube 16 may be connected to compartment 23 so as to define a fixed distance between hub 14 and the compartment. The lumen through inner tube 16 may be sized to slidingly receive a guidewire (not shown) for use in guiding the delivery device to the target site. At its proximal end, inner tube 16 may be provided with a hemostasis valve (not shown) for preventing, or at least hindering, blood flow out from the inner tube.

Hub 14may be adapted for connection to another system or mechanism, such as an operating handle (not shown) for displacing the distal sheath 30. Mechanisms for displacing the distal sheath 30 between its proximal and distal positions are described in International Patent Application Publication No. WO/2009/091509, the disclosure of which is hereby incorporated by reference herein. A retaining ring 15 may be mounted on the inner tube 16 near hub 14.

Catheter assembly 12 may further include an outer shaft 20 which is connected at its distal end through a tapered transition member 24 to the proximal end of distal sheath 30, and at its proximal end to the operating handle (not shown). A Y-connector 18 may also be connected at the proximal end of outer shaft 20, and may include a hemostasis valve for hindering blood flow out from between the inner tube 16 and the outer shaft 20. The Y-connector 18 may also be coupled to a fluid source for flushing the outer shaft 20, injecting contrast media during a prosthetic valve implantation procedure, and the like. The components shown in FIG. 2 may, in some examples, instead be replaced with an operating handle, such as that described in U.S. Patent Publication No. 2024/0180701, the disclosure of which is hereby incorporated by reference herein. For example, the outer shaft 20 may extend distally from the handle, and may be translated proximally or distally relative to the support shaft 28, by rotating a wheel on the handle operably coupled to the outer shaft 20 via a translating carriage, or the like. The support shaft 28 may be an inner or guidewire shaft that also extends into the handle (e.g. the support shaft 28 and inner tube 16 may be part of the same structure). The specifics of the proximal end of the delivery device 10 are not critical to the present disclosure, other than the inclusion of a mechanism to open or close the compartment 23 by withdrawing or advancing the outer shaft 20 (and thus the distal sheath 30) relative to the inner or support shaft 28.

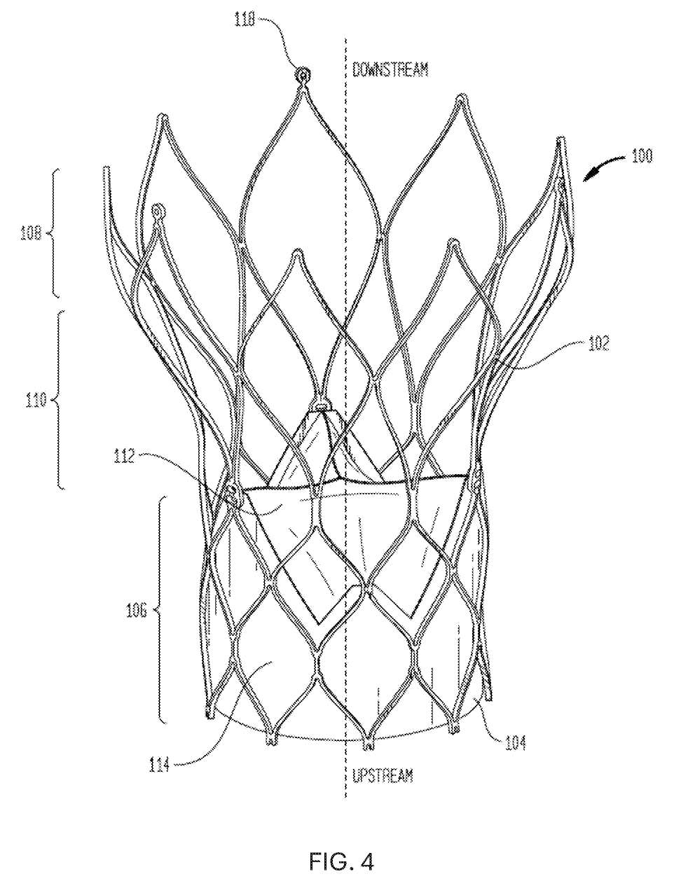

As shown in FIG. 3, the retaining element 26 may include a plurality of recesses 27 located around its periphery. The recesses 27 are spaced apart from one another and each is sized and shaped to receive a tab or retainer on one end of the prosthetic heart valve to maintain the prosthetic heart valve in assembled relationship with the delivery device 10, to minimize longitudinal movement of the prosthetic heart valve relative to the delivery device during unsheathing and resheathing procedures, to help prevent rotation of the prosthetic heart valve relative to the delivery device as the delivery device is advanced to the target site and during deployment, and to maintain the alignment of the stent cells and prevent them from becoming tangled.

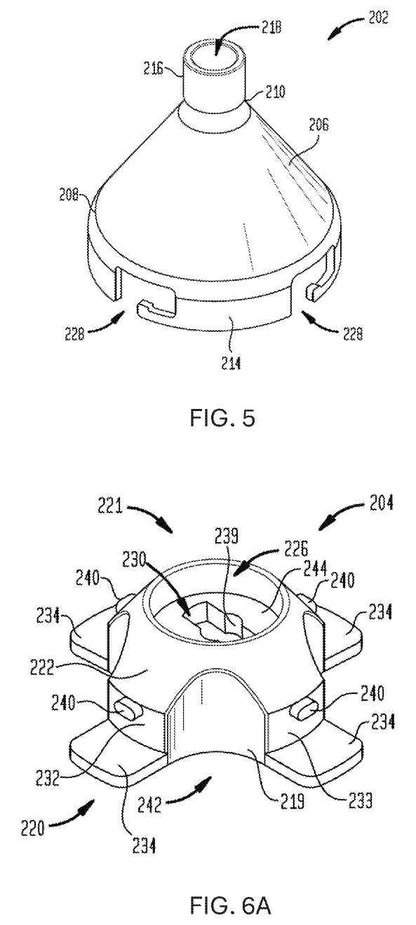

FIG. 4 shows an example of a self-expandable prosthetic heart valve 100 designed to replace a native aortic valve. The valve 100 has a collapsed condition and an expanded condition and may be formed from a collapsible frame or stent 102, with a valve assembly 104 internally connected to the stent. The stent 102 may be formed from any suitable biocompatible material, such as nitinol or any other suitable elastic or shape memory material, and may include an annulus section 106, an aortic section 108, and a transition or sinus section 110 located between the annulus section and the aortic section. The aortic section 108 may have a larger cross section than the annulus section 106. The valve assembly 104 may include a plurality of leaflets 112 and a cuff 114 attached to the interior of the stent 102. The leaflets 112 and the cuff 114 may be formed from a biocompatible polymer, from natural tissue such as bovine or porcine pericardial tissue, or from other appropriate biocompatible materials. The valve assembly 104 may be connected to the stent 102 generally within the annulus section 106. The valve 100 may include a plurality of tabs or retainers 118 at spaced positions around one or both ends of the stent 102 for engagement with the retaining element 26 of the delivery device 10 as described above. The retainers 118 may also be utilized to collapse the valve 100 for loading into the delivery device 10, as will be discussed below.

The valve 100 may be stored in its expanded or open condition. Storing the prosthetic heart valve 100 in its collapsed condition for extended amounts of time may, in some circumstances, increase the likelihood that the tissue leaflets 112 will calcify upon implantation, and thus there may be a preference for prosthetic heart valve 100 to remain in its expanded condition prior to use. In such circumstances, it is necessary to crimp (or otherwise collapse) the valve 100 into a collapsed condition of reduced cross section for loading onto or into the delivery device 10 just prior to the implantation procedure. In order to effectively limit the time period the valve 100 is collapsed, the crimping process may be conducted in the operating arena by the surgeon, interventional cardiologist or other medical or technical personnel using a specialized assembly. It should be understood that, although one particular self-expanding prosthetic heart valve 100 is described herein, it should be understood that the loading devices and methods may generally apply to other self-expanding prosthetic heart valves (or stents) that has retaining systems (e.g. retainers generally similar to retainers 118 configured to interact with recesses of a delivery device generally similar to recesses 27) generally similar to those described herein.

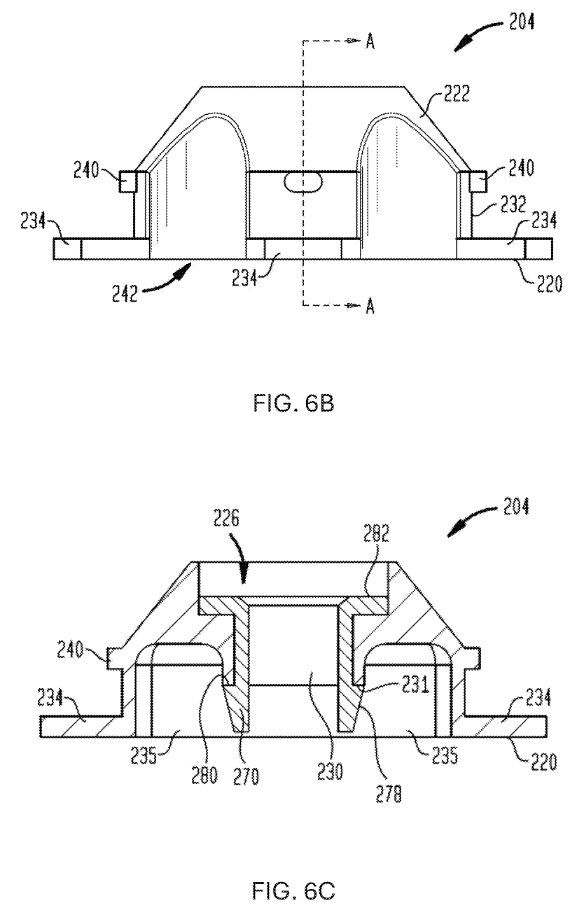

FIGS. 5-8 illustrate components of a loading assembly 200 that generally includes a compression member 202 and a support member 204 adapted to be coupled to one another, a constricting member 300 and a spacer 270. The compression member 202 includes a funnel 206 having a substantially frustoconical shape with a large diameter at a first end 208 and a smaller diameter at a second end 210. The diameter of the funnel 206 may decrease uniformly or non-uniformly from the first end 208 to the second end 210 to compress the valve 100 as it is advanced through the compression member 202. The compression member 202 may be made of a substantially rigid material, and may be wholly or partly made of a transparent plastic, such as polycarbonate or acrylic, to allow viewing of the valve 100 during loading.

The compression member 202 may further include an annular rim 214 extending from the first end 208 of the funnel 206 for joining the compression member to the support member 204. The rim 214 may include a plurality of slots 228 disposed around its outer periphery. While the drawings show slots 228 that are substantially L-shaped or P-shaped, the slots may have any other shapes suitable for securely holding the compression member 202 to the support member 204. The rim 214 may include four such slots 228, or more or fewer than four. Regardless of the number of slots 228, adjacent slots may be spaced equidistantly from each other.

The compression member 202 also may include a tubular extension 216 projecting from the second end 210 of the funnel 206. The tubular extension 216 has an opening 218 therethrough in communication with the interior of funnel 206. The opening 218 is sized and shaped to receive the distal sheath 30 of the delivery device 10 therein. The cross section of the tubular extension 216 is preferably substantially circular, but may be oblong, oval, elliptical, or polygonal.

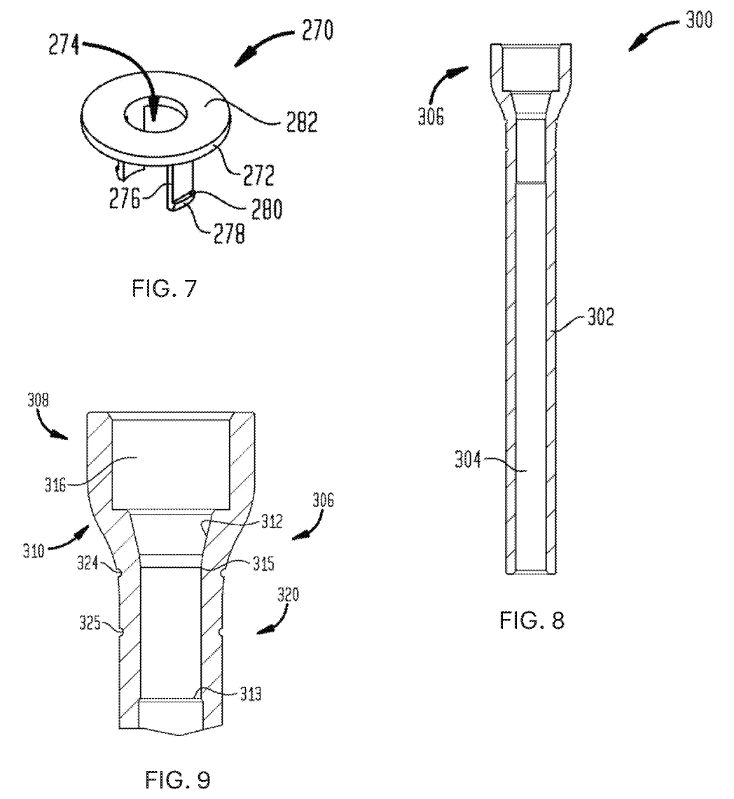

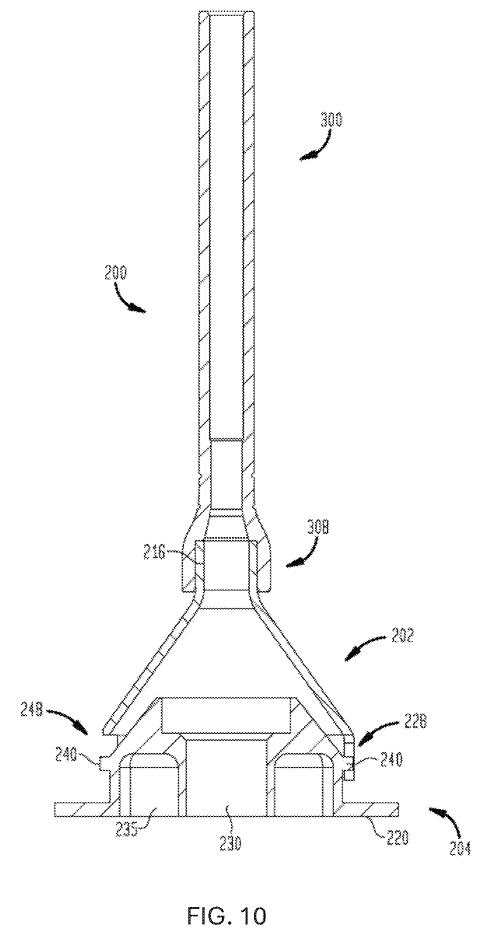

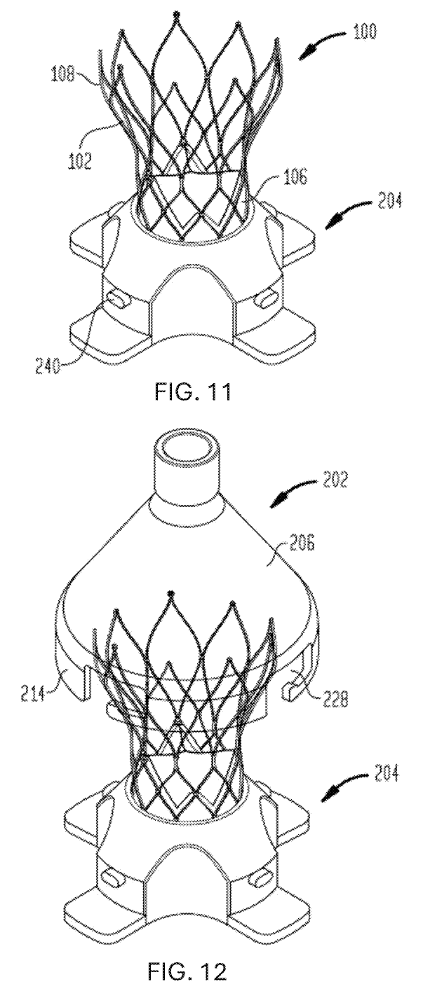

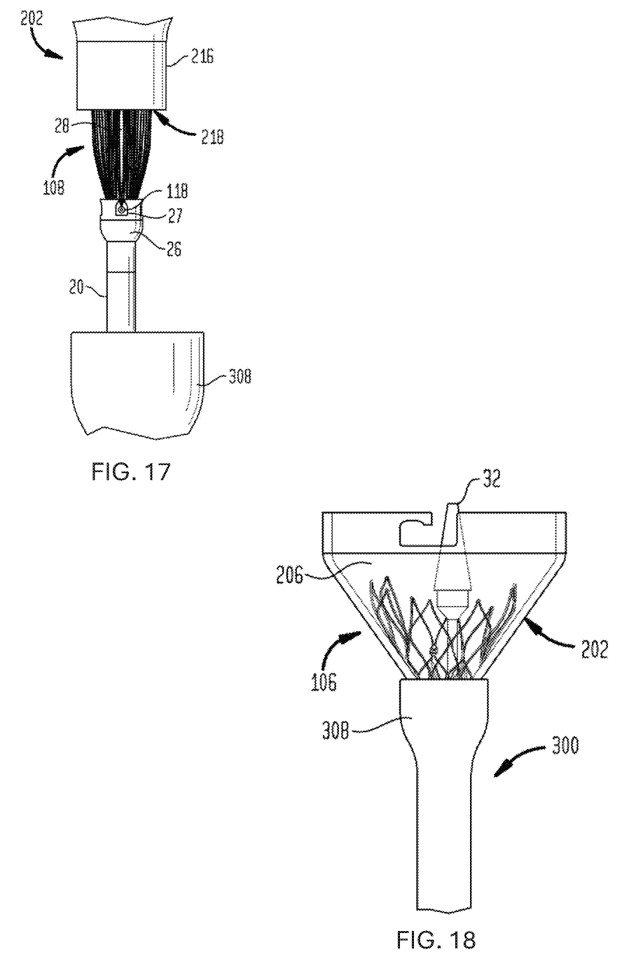

With reference to FIGS. 6A, 6B, 6C and 10, the support member 204 may be made in whole or in part of a substantially rigid material, and includes a body 219 having a substantially flat or planar bottom support surface 220 and a top end 221. Body 219 has an outer wall 232 and a generally rectangular aperture 230 extending therethrough. Aperture 230 has a generally cylindrical central portion 239 sized and shaped to receive at least a portion of the tip 32 of the delivery device 10 therein. A recess 226 extends downwardly from the top end 221 of the body 219 concentrically with bore 230 so as to define a support surface 244 at a spaced distance from the top end. Recess 226 has a diameter and a depth defined by support surface 244 sufficient to receive at least a portion of the annulus section 106 of the stent 102 in an expanded condition.

The outer wall 232 of body 219 does not extend continuously around the body, but rather may be interrupted by a plurality of inwardly curved indentations 242 which divide the outer wall into a plurality of wall segments 233, only two of which are shown in FIG. 6A. Although FIG. 6A depicts a support member 204 having four indentations 242 evenly spaced around the periphery of body 219, it is contemplated that the support member may be provided with more or fewer than four such indentations. Indentations 242 facilitate the grasping of support member 204. Between indentations 242, that is, in the space between outer wall segments 233 and bore 230, body 219 may include a plurality of recesses 235 extending inwardly from the bottom support surface 220. Recesses 235 reduce the mass of body 219 and facilitate the manufacturing process by eliminating excessively thick portions of the body.

The outer wall segments 233 of body 219 do not extend all the way to the top end 221 of the body, but rather terminate at their top ends at a continuous wall 222 oriented at an oblique angle to the outer wall 232. At their bottom ends, outer wall segments 233 each include a radially projecting supporting plate 234, the bottom surfaces of which are substantially coplanar with the bottom support surface 220 of body 219. At least one pin 240 may protrude radially outward from each outer wall segment 233. Pins 240 are preferably spaced a sufficient distance from supporting plates 234 and sized and shaped to be received in the slots 228 of the compression member 202 to join the compression member and the support member 204 together. When joined together, the compression member 202 and the support member 204 collectively define a partial loading assembly 201.

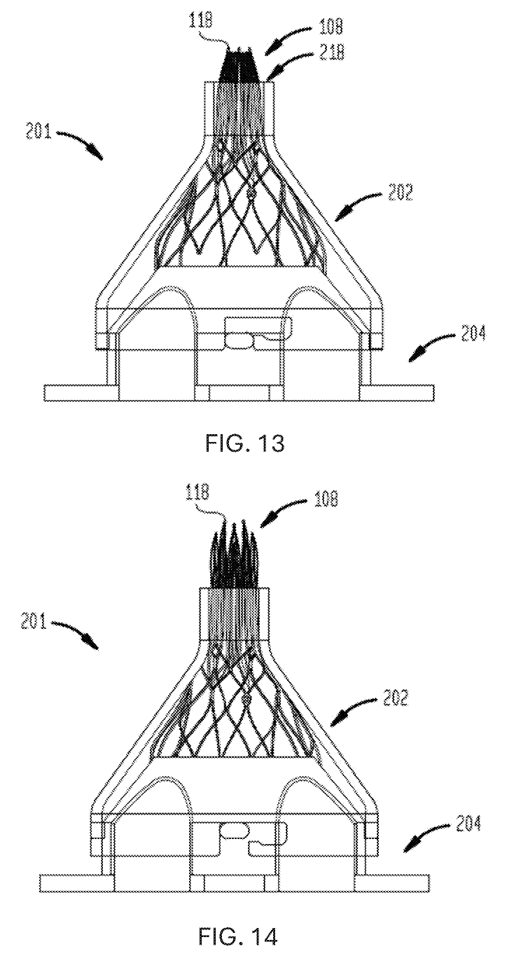

FIG. 7 illustrates a spacer 270 which optionally may be used in connection with support member 204 when a relatively small valve 100 is to be loaded into delivery device 10 using loading assembly 200. Spacer 270 has a generally flat annular portion 272 with a central aperture 274 sized and shaped to receive at least a portion of the tip 32 of the delivery device 10 therethrough. Each leg in a pair of resilient legs 276 has a tapered surface 278 adjacent its free end which terminates at a spaced distance from the free end in a retention surface 280. Spacer 270 may be used to reduce the overall depth of the recess 226 in support member 204. Thus, spacer 270 may be inserted into recess 226 with legs 276 projecting into aperture 230. As spacer 270 is pushed downwardly toward support surface 244, the tapered surfaces 278 adjacent the free ends of legs 276 will contact the narrow sides of aperture 230, causing legs 276 to flex inwardly until retention surfaces 280 engage with a bottom edge 231 of aperture 230 or other structures in the aperture 230 to cause spacer 270 to lock in place. The top surface of annular portion 272 will thus present a new support surface 282 for the annulus section 106 of the stent 102 during a valve loading procedure. The new support surface 282 will be spaced from the support surface 244 of recess 226 by the thickness of annular portion 272. Support surfaces 244 and 282 may be marked with a size or other indicia (not shown) indicating the size of the valve that support surface is intended to be used with.

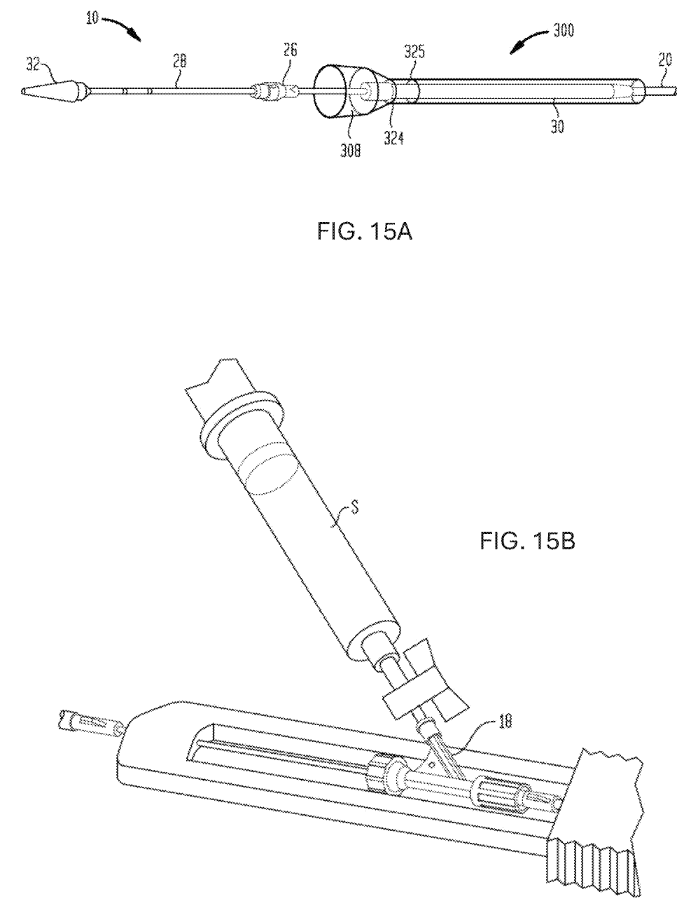

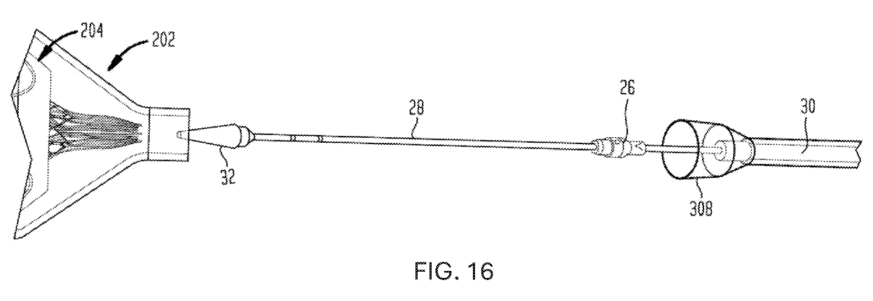

FIGS. 8 and 9 illustrate a constricting member 300 designed to minimize the flaring of the distal end 21 of the distal sheath 30 during loading of a prosthetic heart valve into the compartment 23 of delivery device 10. The constricting member 300 may be wholly or partly made of a transparent plastic, such as polycarbonate or acrylic, to allow viewing of the delivery device 10 during loading and includes a tubular member 302 having a central lumen 304 sized and shaped to slidingly receive at least the distal sheath 30 of the delivery device 10.

As shown in FIG. 9, at one end 306, the constricting member 300 may have an enlarged head 308 with a counterbore 316 formed therein. The counterbore 316 may have a diameter that is larger than the diameter of lumen 304, and in particular, may be sized and shaped to receive the tubular extension 216 of the compression member 202. Preferably, the diameter of counterbore 316 is only slightly larger than the outer diameter of the tubular extension 216 so as to create a friction fit therebetween.

Between the tubular member 302 and the enlarged head 308, constricting member 300 may have a tapered portion 310. In particular, tapered portion 310 may have an inner surface 312 which tapers from a larger diameter at its end adjacent the counterbore 316 to a smaller diameter at its other end to help compress valve 100 further during loading into delivery device 10.

The constricting member 300 may further include a transition portion 320 disposed between the tapered portion 310 and the tubular member 302. The transition portion 320 may have a substantially constant inner diameter sized and shaped to receive at least the distal sheath 30 of the delivery device 10. The inner diameter of the transition portion 320 may be slightly smaller than the diameter of lumen 304 and slightly larger than the outer diameter of the distal sheath 30 in order to substantially prevent or minimize the flaring of the distal end 21 of the distal sheath 30 while the valve 100 is loaded in the delivery device 10, as discussed in detail below. The larger diameter of the lumen 304 allows a user to easily slide the constricting member 300 over the distal sheath 30 of the delivery device 10. In a variant hereof, the transition portion 320 may have an inner diameter which tapers downwardly from a slightly larger diameter at an end 313 thereof to a slightly smaller diameter at an end 315 thereof to accommodate small variations in the outer diameter of the distal sheath 30.

An annular groove or other indicator line 324 may extend partly or entirely around the outer periphery of the tubular member 302 at the junction between the tapered portion 310 and the transition portion 320. Another annular groove or indicator line 325 may extend partly or entirely around the outer periphery of the tubular member 302 at a spaced distance from the first line 324. Lines 324 and 325 mark the area in which the user should place the distal end 21 of the distal sheath 30 during the loading procedure. As discussed in detail below, using the constricting member 300 to help load the valve 100 into the delivery device 10 reduces the loading forces (i.e., the forces required to load the valve into the delivery device) and reduces flaring of the distal end 21 of the distal sheath 30.

FIG. 10 shows an assembled loading assembly 200 including the compression member 202 of FIG. 5, the support member 204 of FIG. 6 and the constricting member 300 of FIG. 8. As shown in FIG. 10, the constricting member 300 is connected by its enlarged head 308 to the tubular extension 216 of the compression member 202, and the compression member 202 is locked to the support member 204. To lock the compression member 202 to the support member 204, the pins 240 of the support member are inserted into the slots 228 of the compression member, and the compression member is turned relative to the support member to slide the pins toward the closed ends of the slots. Hence, the pins 240 and the slots 228 together form a locking mechanism 248. Rather than the engagement of the pins 240 in the slots 228, it is contemplated that any other known locking mechanisms may be employed to securely lock the compression member 202 to the support member 204.

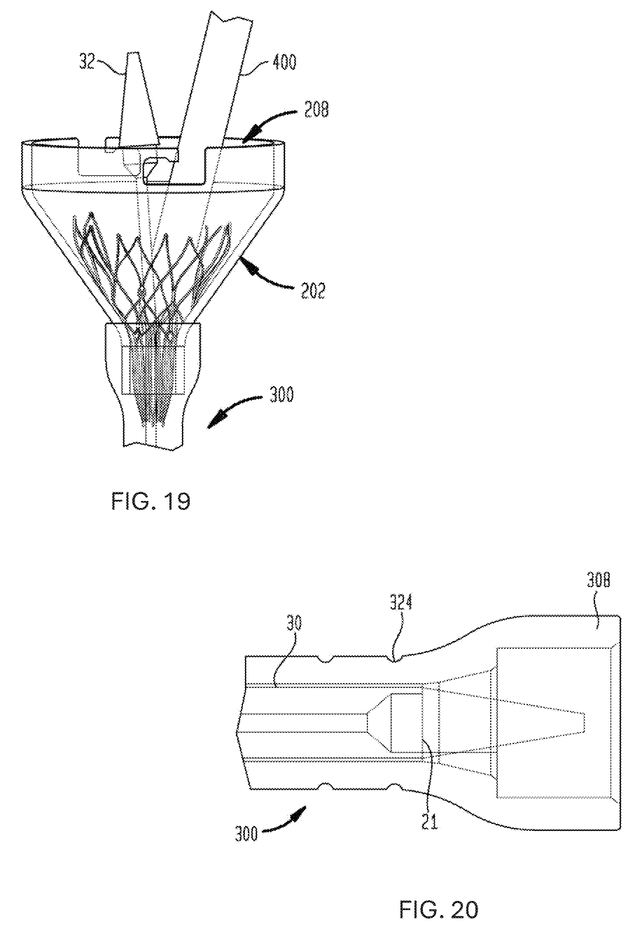

As shown in FIG. 11-20, the loading assembly 200 may be used to load the collapsible prosthetic heart valve 100 into a delivery device 10. Where a relatively small heart valve 100 is to be implanted, the spacer 270 is first assembled in the recess 226 of the support member 204 to decrease the overall depth of the recess. Then, as shown in FIG. 11, with the support member 204 on a flat surface, at least a portion of the annulus section 106 of the stent 102 may be placed within the recess 226 of the support member until the end of the stent contacts support surface 282 on spacer 270. The compression member 202 may then be placed over the aortic section 108 of the stent 102 so that the aortic section of the stent is positioned within the funnel 206, as depicted in FIG. 12. As shown in FIG. 13, the compression member 202 and the support member 204 may then be pushed together, the tapered walls of the funnel 206 gradually compressing the valve 100 until a portion of the aortic section 108 of the stent 102 is forced into and through the opening 218 of the compression member. When a portion of the aortic section 108 of the stent 102 passes through the opening 218 of the compression member 202, the retainers 118 of the stent will protrude through the opening 218 and will be positioned closely adjacent to one another. Even though the heart valve being compressed is relatively small, the use of spacer 270 reduces the overall depth of recess 226 such that the retainers 118 of stent 102 will protrude from opening 218 when the stent is collapsed. At this point, the pins 240 of the support member 204 will be positioned within the slots 228 of the compression member 202, and the members may be locked together by rotating the support member relative to the compression member, such that the pins 240 of the support member slide toward the closed ends of the slots 228 of the compression member.

As shown in FIG. 15A, with the distal sheath 30 in a proximal or open position, the constricting member 300 may be placed over the delivery device 10 with the enlarged head 308 positioned closer to the tip 32 than to the hub or handle of the delivery device, and with the distal end 21 of the distal sheath 30 longitudinally positioned between indicator lines 324 and 325 of the constricting member. It will be appreciated that the constricting member 300 also may be placed over the delivery device 10 with the distal sheath 30 in the distalmost or closed position, and that the distal sheath subsequently may be moved to the proximal or open position.

Before loading the valve 100 into the delivery device 10, it may be preferable to subject the delivery device to a deairing process. In that regard, with the constricting member 300 assembled over the distal sheath 30 and the distal sheath in an open position, a syringe S may be connected to the Y-connector 18 of the delivery device 10, as shown in FIG. 15B. The syringe may be used to inject a sterile liquid, such as saline, into the proximal end of the delivery device and out through the open compartment 23, thereby flushing the air from the device. During this flushing step, the distal end of the delivery device may be tapped multiple times to facilitate the air removal.

Once flushing of the delivery device 10 has been completed, the tip 32 and the support shaft 28 of the delivery device 10 may be inserted into the end of the collapsed valve 100 protruding from the opening 218 of the compression member 202. To accomplish this, the compression member 202 and the support member 204 may be squeezed closer together. (The dimension of the slots 228 in the longitudinal direction, i.e., the height of the slots, is greater than the dimension of the pins 240 in the longitudinal direction, i.e., the height of the pins. Therefore, even though the compression member 202 and the support member 204 are assembled together, they still may move further toward one another.) As the compression member 202 and the support member 204 move closer together, a greater portion of the stent 102 is forced out through opening 218, causing the retainers 118 to begin to separate from one another, as illustrated in FIG. 14. The tip 32 and support shaft 28 of the delivery device 10 may then be inserted between the retainers 118 and into the end of the collapsed valve 100, as shown in FIG. 16. The partial loading assembly 201 then may be advanced along the support shaft 28 until the retainers 118 of the stent 102 are positioned over the retaining element 26 of the delivery device 10. The partial loading assembly 201 may be twisted as needed to align the retainers 118 with the recesses 27 in the retaining element 26. Positioning the retainers 118 within the recesses 27 of the retaining element 26 attaches the stent 102 to the delivery device 10, as seen in FIG. 17. With the stent 102 attached to the retaining element 26, the constricting member 300 and the distal sheath 30 may be slid together toward the partial loading assembly 201 (or the inner tube 16 may be moved proximally relative to the constricting member 300 and the distal sheath 30) to about the position shown in FIG. 18, in which the distal sheath covers the retainers 118 of the stent, at the same time maintaining the distal end 21 of the distal sheath between indicator lines 324 and 325. The tapered inner surface 312 of the enlarged head 308 facilitates the compression of the stent 102 as it moves into the constricting member 300. When the constricting member 300 and the partial loading assembly 201 are close together, they may be joined to one another by assembly of the enlarged head 308 of the constricting member 300 to the tubular extension 216 of the compression member 202.

In order to deair the valve 100, a sterile liquid, such as saline, may be dispensed into the compression member 202 through its first open end 208. To do so, the support member 204 may be disassembled from the compression member 202 by first rotating the support member relative to the compression member, such that the pins 240 of the support member slide toward the open ends of the slots 228 of the compression member. This action unlocks the members from one another. The support member 204 may then be moved away from the compression member 202 to disassemble the partial loading assembly 201. With the first open end 208 of the funnel 206 facing up, the sterile liquid may be dispensed into the compression member 202 through the first open end. The sterile liquid may be dispensed into the compression member 202, such as through a syringe or a sterile container, until the funnel 206 is substantially filled, as shown in FIG. 18. The syringe may need to be refilled several times during the injection process in order to fill the funnel 206 with the sterile liquid.

Any air bubbles in the sterile liquid within the funnel 206 may then be removed. It is preferable that little or no air be released into the human body during deployment and/or re-sheathing of the valve within the human heart, as the air may block vascular flow and cause tissue damage. For this reason, it is preferable to remove air bubbles from the delivery device 10 and the valve 100 before introducing them into the body. Testing has shown that, if the methods and assemblies described in this application are employed, minimal air will be released into the patient’s bloodstream during valve deployment and re-sheathing.

Air bubbles formed in the sterile liquid near the space between the leaflets 112 and the cuff 114 of the valve 100 may be removed by using a tube or rod 400 or any other suitable atraumatic probe. The tube 400 is commonly known in the art as a “leaflet tester” and may be formed of a substantially soft material, such as a soft polymer. In order to remove the air bubbles from the sterile liquid, the tube 400 may be placed into the sterile liquid contained in the funnel 206 of the compression member 202 and used to probe areas of potential air entrapment, including gently agitating the liquid, as shown in FIG. 19. A syringe may be used to remove the air bubbles from the space near the retaining element 26 of the delivery device 10. To do so, the syringe may be inserted into the space near the retaining element 26, and the sterile liquid near the retaining element 26 may be gently agitated with the syringe. After the air bubbles have been removed, the valve 100 may be pulled into the distal sheath 30 until the valve is completely covered, as seen FIG. 20. The constricting member 300 and the compression member 202 may then be removed from the delivery device 10. The inner tube 16 of the delivery device 10 may then be flushed with any suitable sterile liquid using, for example, a syringe. To flush the inner tube 16, a syringe may be connected to the hemostatic valve near the hub 14 of the delivery device 10, and then sterile liquid may be injected into the inner tube using the syringe

In an alternate method of loading the valve 100 into the delivery device 10 and preparing same for use in a patient, the air bubbles may be removed from the distal sheath 30 by submerging the distal sheath, the compression member 202, and the constricting member 300 in a container holding sterile liquid, such as saline. Additional sterile liquid may be injected into the delivery device 10 through the Y-connector 18 using a syringe, as discussed above. The distal sheath 30 of the delivery device 10 may then be shaken and gently tapped against a hard surface to remove air bubbles from the valve 100. The valve 100 may then pulled into the distal sheath 30, as discussed above.

In view of the tight fit between the collapsed valve 100 and the distal sheath 30, significant friction forces may need to be overcome in order to move the distal sheath 30 completely over the valve 100. To facilitate this procedure, the stent 102 may be substantially cooled, which, depending on the materials forming the stent, may enable the stent to more easily deform. Thus, once more than about one-half of the length of the stent 102 has been covered by the distal sheath 30, a cold liquid, such as saline solution, may be applied to the stent through the compression member 202 and the constricting member 300. This may be accomplished by removing the support member 204 from the compression member 202 and holding the remainder of the assembly in a substantially vertical orientation with the first end 208 of the funnel 206 facing upwardly. The cold liquid may then be introduced into the compression member 202 using any suitable apparatus. It will, of course, be appreciated that the cold liquid may thus serve two purposes — it may cool the stent 102, and it may serve as the deairing liquid in the deairing procedure described above.

In order for the cooling of the stent 102 to be effective in making it easier for the stent to be completely covered by the distal sheath 30 of the delivery device 10, the stent should be cooled to a temperature below the transition temperature of the material forming the stent. The “transition temperature” of a material is the temperature at which the material changes from one crystal state to another. For the nitinol stents that may be employed in the present invention, a saline solution at about 0° C. may be used. When cooled below its transition temperature, the stent 102 becomes plastic, enabling it to deform much more readily under the forces exerted by the movement of the distal sheath 30 thereover. Accordingly, after the stent 102 has been cooled below the transition temperature, the user may completely cover the stent 102 with the distal sheath 30 of the delivery device 10, as illustrated in FIG. 20.

The distal sheath 30 of the delivery device 10 should be non-traumatic. To accomplish this, the distal sheath 30 may be made of soft polymeric material. However, while the valve 100 is loaded into the delivery device 10, the distal end 21 of the distal sheath 30 may slightly expand or flare due to the pressure exerted by the self-expanding stent 102. Since the distal sheath 30 is typically formed from a soft polymer, the distal end 21 of the distal sheath may not return to its original shape once the distal sheath completely covers the valve 100. It is nonetheless important to maintain the original cross-sectional profile of the distal end 21 of the distal sheath 30, because doing so makes the distal sheath more atraumatic and reduces the loading forces required to load the valve 100 into the delivery device 10. In order to maintain the original circular profile of the distal end 21 of the distal sheath 30, the loading assembly 200 preferably includes the constricting member 300 described above.

The present disclosure contemplates that the delivery device 10 and the loading assembly 200 may be provided together in the form of a kit. Thus, the kit may include a delivery device 10 for delivering the heart valve into the patient, as well as a loading assembly 200 for loading the heart valve into the delivery device. The loading assembly 200 would include all of the components necessary to load a heart valve into the delivery device, regardless of the size of heart valve to be deployed. In other words, the loading assembly 200 would include a compression member 202, a support member 204, and a constricting member 300, as well as a spacer 270. In cases in which the heart valve to be deployed is relatively large, the spacer would not be used to reduce the depth of the recess 226 in support member 204. However, where the heart valve to be deployed is relatively small, spacer 270 may be used to assure that the retainers 118 of the heart valve stent 102 protrude through the opening 218 of compression member 202, as shown in FIG. 14. It will be appreciated that, where there is a large range in heart valve sizes that may be deployed by delivery device 10, the kit may include multiple spacers 270, each spacer being sized for use with one or more heart valve sizes, the support surface or other surface of each spacer being marked with an indicia of the valve size that spacer is intended to be used with. Alternatively, the spacer could be color-coded for use with one or more heart valve sizes. In that regard, the thickness of annular portion 272 may differ from one spacer 270 to another so as to reduce the depth of recess 226 by an appropriate amount to accommodate the size of the particular heart valve being loaded into delivery device 10.

As should be understood from the above disclosure, it is important for the retainers 118 to be properly positioned within their corresponding recesses 27 to help ensure (i) proper loading of the prosthetic heart valve 100 into the capsule 23 without damaging the prosthetic heart valve 100, (ii) proper maintenance of the prosthetic heart valve 100 relative to the delivery device 10 during deployment, particularly before complete release, and (iii) proper full release of the prosthetic heart valve 10 from the delivery device 10 when the distal sheath 30 is withdrawn proximally of the retainers 118. Referring back to FIG. 17, when loading prosthetic heart valve 100 into the delivery device 10 using loading assembly 200, there is a point during the loading in which the retainers 118 are manually placed into their corresponding recesses 27, and the head 308 of the constricting member 300 still needs to be pushed distally to engage with the tubular extension 216 of the compression member 202. Particularly during this step, there is a possibility that the retainers 118 may disengage from their respective recesses 27 as the constricting member 300 is advanced distally over the retainers 118. If this occurs, misload of the retainers 118 may occur, in which some or all of the retainers 118 are not seated (or not fully and/or properly seated) within their corresponding recesses 27. If this occurs, it may not be obvious to the user that a misload has occurred, particularly of the constricting member 300 is not transparent. Thus, the user may not realize that the retainers 118 are not going to be able to perform their intended function until deployment of the prosthetic heart valve 100 begins, at which point it may be too late to perform any corrective action, for example if the prosthetic heart valve 100 prematurely fully deploys from the delivery device 10 in a sub-optimal position within the patient. Loading assembly 1200, described in greater detail below, may help mitigate at least some of the risks described in this paragraph. It should be understood that loading assembly 1200 generally follows the numbering provided in connection with loading assembly 200, except each part number is provided with a leading 1.

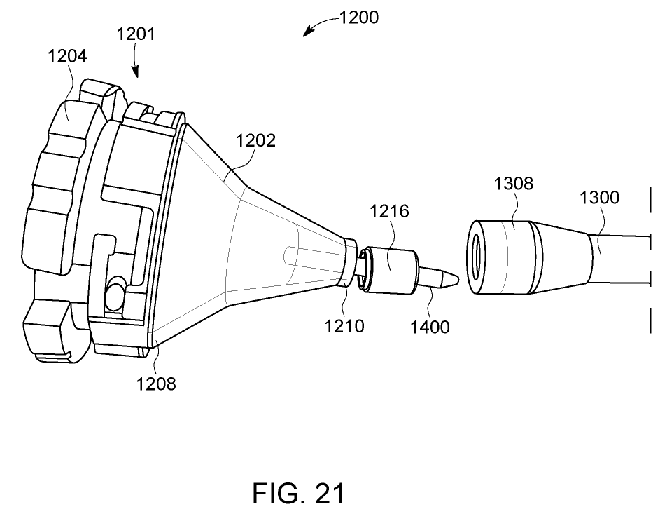

FIG. 21 shows loading assembly 1200, including a compression member 1202 already assembled to a support member 1204 to form a partial loading assembly 1201, with a head 1308 of a constricting member 1300 positioned adjacent to the compression member 1202. FIG. 21 also shows a shaft or stick 1400 protruding through the compression member 1202. It should be understood that support member 1204 may be similar or identical to support member 204 in structure and function, and is thus not described in further detail herein. Also, constricting member 1300 may be similar or identical to constricting member 300 in structure and function, other than differences described herein. Finally, the compression member 1202 may be similar in structure and function to compression member 202, with the main difference being the inclusion of a separate locking ring 1216 (e.g. instead of or in addition to an integral tubular extension 216). Thus, other than the structures and functions of loading assembly 1200 shown or explicitly described herein, the structure and function of loading assembly 1200 may be similar or identical to the corresponding structure and functions loading assembly 200, described in more detail above.

Still referring to FIG. 21, compression member 1202 may have a similar shape to compression member 202, including a conical or frustoconical shape extending from a first end 1208 with a large diameter to a second end 1210 with a smaller diameter. However, unlike the compression member 202, the compression member 1202 may end relatively abruptly at the second end 1210, or include a small extension that is significantly shorter compared to tubular extension 216. Rather, instead of having a tubular extension similar to tubular extension 216, the loading system 1200 includes a ring 1216, which may be referred to as a locking ring 1216. Locking ring 1216 may be cylindrical, having a shape and size generally similar to tubular extension 216, but locking ring is not fixedly or integrally coupled to the second end 1210 of compression member 1202. In some embodiments, the locking ring 1216 may have an axially tapered internal diameter. Preferably, the inner diameter of the locking ring 1216 (or the smallest inner diameter of the locking ring 1216 if the locking ring is tapered) is about equal to, or only slightly larger than, the outer diameter of the retainer 26. Locking ring 1216 is preferably transparent. It may also be preferable for the compression member 1202 and/or the constricting member to be transparent. Support member 1204 may also be transparent.

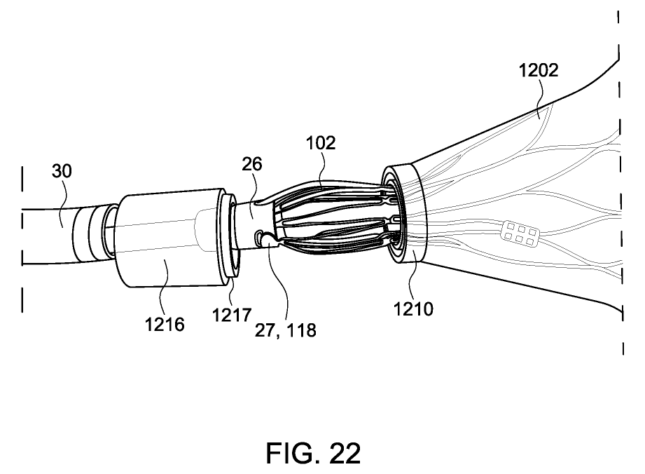

The locking ring 1216 may include an inner diameter that is about equal to, or slightly larger than, the outer diameter of the retaining element 26 (e.g. the portion of the retaining element directly circumferentially adjacent the recesses 27). As best shown in FIG. 22, one end of the locking ring 1216 may include a rim 1217, which may have a diameter smaller than the outermost diameter of the locking ring 1216, configured to be received snugly within the second end 1210 of compression member 1202 (or within a corresponding annular groove formed in the second end 1210 of compression member 1202). However, this is just one example, and other example connections, including a snap-fit connection (or even a connection similar to how the support member 1204 couples to the compression member 1202) may be used. It should thus be understood that any connection mechanism may be suitable.

Loading assembly 1200 may be used in a largely similar fashion as described in connection with loading assembly 200. For example, similar to as shown in FIGS. 11-14, the prosthetic heart valve 100 may be placed into support member 1204 (with or without a corresponding spacer similar or identical to spacer 270), the large end 1208 of the compression member 1202 may be placed over the prosthetic heart valve and pushed down over the prosthetic heart valve 100 to partially collapse the prosthetic heart valve 100, and the compression member 1202 may be rotated to engage pins of the support member 1204 with corresponding slots of the compression member 1202. At this stage, the loading assembly 1200 and prosthetic heart valve 100 may look substantially similar to the configuration shown in FIG. 14, with the exception that more of the prosthetic heart valve 100 may extend from the smaller end 1210 of the compression member 1202 due to the absence of an extension similar to tubular extension 216. Although not described in connection with FIGS. 11-14, the shaft or stick 1400 may extend through the interior of the prosthetic heart valve 100 and the compression member 1202 up to the corresponding stage shown in FIG. 14, at which point the shaft or stick 1400 may be removed. Prior to removal, the shaft or stick 1400 may help prevent the retainers 118 from crossing under each other (e.g. by serving as a physical barrier) as the outflow end of the prosthetic heart valve 100 collapses and the retainers 118 draw more closely together.

As part of the loading method, the constricting member 1300 may be placed over the delivery device 10 in substantially the same fashion as shown and described in connection with FIG. 15A. The locking ring 1216 may also be placed over the delivery device, for example positioned between the retaining element 26 and the enlarged head 1308 of the constricting member 1300, with the annular rim 1217 facing toward the atraumatic distal tip 32 of the delivery device 10. It should be understood that deairing is not described in detail in connection with the use of loading assembly 1200, but deairing may be performed similarly or identically to as described above in connection with loading assembly 200. As part of the deairing process, the support member 1204 may be disassembled from the compression member 1202 and put to the side for the remainder of the loading procedure.

At this point, the tip 32 and support shaft 28 of the delivery device 10 may then be inserted between the retainers 118 and into the end of the collapsed valve 100, in substantially similar fashion as shown in FIG. 16. The partial loading assembly 1201 then may be advanced along the support shaft 28 until the retainers 118 of the stent 102 are positioned over the retaining element 26 of the delivery device 10. As shown in FIG. 22, at this point, the retainers 118 maybe positioned or received within their corresponding recesses 27 on retaining element 26, while the locking ring 1216 is positioned between the distal end of the distal sheath 30 and the retaining element 26.

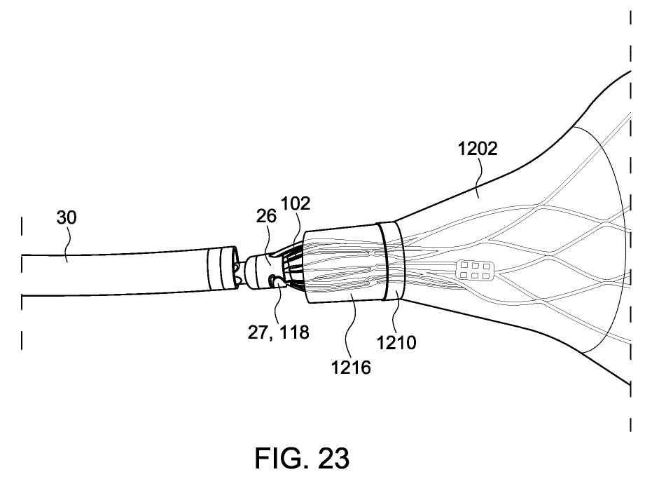

After achieving the configuration shown in FIG. 22, the user may advance the locking ring 1216 until it is positioned directly over the retainers 118 and the retaining element 26. As noted above, the interior diameter of the locking ring 1216 may be about equal to, or slightly larger than, the outer diameter of the retaining element 26. Thus, when the locking ring 1216 is over the retaining element 26 (and the retainers 118 received within their corresponding recesses 27), the retainers 118 are held within their corresponding recesses 27 by the overlying locking ring 1216. Further, if the locking ring 1216 is transparent, the user can visualize each retainer 118 to confirm each retainer 118 is properly received within its corresponding recess 27 of retaining element 26. The annular rim 1217 of the locking ring 1216, at this point, may fit snugly within the second end 1210 of the compression member 1202 so that the locking ring 1216 is temporarily coupled (e.g. by snap fit or interference fit) to the compression member 1202. After this step, the loading system 1200 may have a configuration substantially similar to that shown in FIG. 23. However, it should be understood that FIG. 23 shows the locking ring 1216 a spaced distance from retainers 118. In some examples, the locking ring 1216 may be positioned directly over the retainers 118. However, in other examples, the locking ring 1216 may be placed over the portion of the stent 102 adjacent to the retainers 118. In either configuration, the locking ring 1216 will maintain the retainers 118 within their corresponding recesses 27 with the ability to clearly visualize the retainers 118 to confirm they remain in the desired position. In some examples, instead of an annular rim 1217, the locking ring 1216 may have a threaded end that can threadedly engage the smaller diameter end 1210 of the compression member 1202.

At this point, the loading assembly 1200 may have a similar configuration as shown in FIG. 17, with the exception that the locking ring 1216 may be positioned directly over (or directly adjacent to) the retainers 118, compared to the configuration shown in FIG. 17 in which the tubular extension 216 is a more significant spaced distance from the retainers 118. Although the constricting member 1300 is not shown in FIG. 23, it would be in substantially the same position at this point as shown in FIG. 17 for constricting member 300 (e.g. with the head 1308 positioned a spaced distance from the retainers 118).

With the stent 102 attached to the retaining element 26, the constricting member 1300 and the distal sheath 30 may be slid together toward the partial loading assembly 1201 (or the inner tube 16 may be moved proximally relative to the constricting member 1300 and the distal sheath 30) until the enlarged head 1308 of the constricting member 1300 is positioned over the locking ring 1216, which may be generally similar to the position shown in FIG. 18. Deairing may be performed, for example in a similar manner as described above in connection with FIGS. 18-19, and the valve 100 may be pulled into the distal sheath 30 until the valve 100 is completely covered. 20. The constricting member 1300 and the compression member 1202 (along with the locking ring 1216 which may still be coupled to the compression member 1202) may then be removed from the delivery device 10. At this point, the prosthetic heart valve 100 is loaded within the compartment 23 in a collapsed condition for delivery, with the distal end of the distal sheath 30 in abutment with the atraumatic distal tip 32, with confidence that the retainers 118 have been properly maintained within their corresponding recesses 27 of the retaining element 26 without any occurrence of a misload.

It should be understood that, compared to loading system 200, the use of transparent locking ring 1206 with loading system 1200 allows the user to verify that the retainers 118 have all been properly seated in their corresponding recesses 27 of the retaining element 26 prior to drawing the prosthetic heart valve 100 into the distal sheath 30 so that misload can be identified early and prevented. Also, because the locking ring 1206 nests into a corresponding pocket within the head 1308 of the constricting member 1300 during loading, the locking ring 1206 does not need to be removed to fully load the valve 100 into the distal sheath 30.

It should further be understood that this same functionality is not achievable with compression member 202, nor would a modification of compression member 202 to have a longer tubular extension 216 allow the same functionality to be achieved as the function achieved by a separate locking ring 1216 and truncated compression member 1202. For example, referring to the configuration shown in FIG. 17, the open end of the tubular extension 216 must be a meaningful spaced distance from the retainers 118 in order to allow the user to physically manipulate the retainers 118 so that they can be manually placed into the recesses 27. The stent 102 is already angled, via partial compression by the compression member 202, so that the retainers 118 are angled inwardly toward each other after passing through the open end of the tubular extension 216. Thus, lengthening the tubular extension 216 so that it extends to cover the retainers 118 is not a solution, as it would make it extremely difficult or impossible to manipulate the retainers 118 as required. That spaced distance described earlier in this paragraph may also be needed to allow room for the stent of the prosthetic heart valve 100 to expand, which allows the user to comfortably feed the atraumatic distal tip 32 through the stent of the prosthetic heart valve 100 prior to engaging the retainers 118 into their recesses 27. However, by having a separate locking ring 1216 and omitting the tubular extension as an integral piece of the compression member 1202, good clearance can be maintained between the retainers 118 (when they protrude from the smaller end 1210 of the compression member 1202) and the recesses 27 of the retaining element 26 for that initial engagement of the retainers 118 into the recesses 27. Only after this engagement is achieved with the available clearance, the locking ring 1216 can come from the other side to lock the retainers 118 into place for the remaining process of loading the valve 100 into the distal sheath 30. It should further be noted that, while the transparency of that locking ring 1216 is useful in helping to visually confirm the proper engagement, the overall enhanced functionality of the loading system 1200 (compared to the loading system 200) would still be achieved even if the locking ring 1216 were not transparent. It should be understood that the locking ring 1216 may also increase radial compression on the stent in order to “lock” the retainers 118 into their corresponding pockets or recesses 27. Typically, with systems such as that shown in FIG. 17, there is some “play” between the retainers 118 within the recesses 27 due the compression member 202 (e.g. at tubular extension 216) having a larger inner diameter than the retainer 26. However, the locking ring 1216 may have an inner diameter that is about the same as, or only slightly larger than, the outer diameter of the retainer 26. With this configuration, any such “play” between the retainers 118 and their corresponding pockets 27 is substantially minimized or eliminated.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of loading a self-expanding prosthetic heart valve into a delivery device, the method comprising:
advancing the prosthetic heart valve through a first end of compression member until a first retainer of the prosthetic heart valve protrudes from an open second end of the compression member, the compression member tapering from a larger diameter at the first end to a smaller diameter at the second end, the first retainer being formed at an end of a stent of the prosthetic heart valve;
while the first retainer protrudes from the open second end of the compression member, advancing a tip of the delivery device through the stent of the prosthetic heart valve until a retaining element of the delivery device is positioned adjacent to the first retainer;
while the retaining element of the delivery device is positioned adjacent to the first retainer, positioning the first retainer within a first recess formed in the retaining element;
while the first retainer is positioned within the first recess, advancing a locking ring from a first position on the delivery device in a distal direction toward the tip of the delivery device to a second position in which the locking ring overlies either (i) the first retainer or (ii) a portion of the stent adjacent to the first retainer, so that the locking ring locks the first retainer within the first recess; and
moving a distal sheath of the delivery device from a proximal open position to a distal closed position, whereby moving the distal sheath results in the prosthetic heart valve being maintained in a collapsed condition within a compartment defined between the tip and the retaining element.

2. The method of claim 1, wherein the locking ring is transparent, and the method includes visually confirming that the first retainer is properly positioned within the first recess while the locking ring overlies either (i) the first retainer or (ii) the portion of the stent adjacent to the first retainer.

3. The method of claim 1, wherein advancing the locking ring from the first position to the second position includes coupling the locking ring to the second end of the compression member.

4. The method of claim 3, wherein the locking ring includes an annular rim, and wherein coupling the locking ring to the second end of the compression member includes achieving a friction fit, a snap fit, or an interference fit between the annular rim and the second end of the compression member.

5. The method of claim 1, further comprising, prior to advancing the tip of the delivery device through the stent of the prosthetic heart valve, positioning a constricting member over the delivery device, so that an enlarged head at an end of the constricting member faces toward the tip of the delivery device.

6. The method of claim 5, further comprising advancing the constricting member until the locking ring is received within the enlarged head of the constricting member while the locking ring overlies either (i) the first retainer or (ii) the portion of the stent adjacent to the first retainer.

7. The method of claim 5, wherein the constricting member is transparent.

8. The method of claim 1, wherein the compression member is transparent.

9. The method of claim 1, wherein the step of advancing the prosthetic heart valve through the first end of compression member is performed after the prosthetic heart valve is positioned, while in an expanded condition, on a support member.

10. The method of claim 9, wherein the step of advancing the prosthetic heart valve through the first end of the compression member includes advancing the support member toward the compression member until the support member contacts first end of the compression member, and the method further includes reversibly coupling the support member to the compression member.

11. A system comprising:
a self-expanding prosthetic heart valve including a stent, a valve assembly supported by the stent, and a first retainer at one end of the stent, the prosthetic heart valve having an expanded condition and a collapsed condition;
a delivery device including a tip, a retaining element having a first recess sized and shaped to receive the first retainer therein, a compartment at least partially defined between the tip and the retaining element and adapted to receive the prosthetic heart valve, and a distal sheath movable between a distal closed position fully covering the compartment and a proximal open position uncovering the compartment, the distal sheath configured to maintain the prosthetic heart valve in the collapsed condition in the compartment when the distal sheath is in the distal closed position;
a compression member having a first open end with a first diameter, a second open end with a second diameter less than the first diameter, and a wall decreasing in diameter from the first open end to the second open end, the wall defining an open space adapted to receive the prosthetic heart valve, the compression member configured to radially compress the prosthetic heart valve as the prosthetic heart valve advances through the open space from the first open end toward the second open end; and
a locking ring having an inner diameter corresponding to an outer diameter of the retaining element, the locking ring configured to slide over the delivery device and to overlie either (i) the first retainer or (ii) a portion of the stent adjacent to the first retainer while the first retainer is received within the first recess so that the locking ring locks the first retainer within the first recess.

12. The system of claim 11, wherein the prosthetic heart valve includes second and third retainers at the one end of the stent.

13. The system of claim 12, wherein the retaining element has a second recess sized and shaped to receive the second retainer therein, and a third recess sized and shaped to receive the third retainer therein.

14. The system of claim 11, wherein the locking ring is transparent.

15. The system of claim 11, wherein the locking ring is configured to reversibly couple to the second open end of the compression member.

16. The system of claim 15, wherein the locking ring includes an annular rim configured to achieve a friction fit, a snap fit, or an interference fit with the second open end of the compression member.

17. The system of claim 11, further comprising a constricting member having an enlarged head at an end of the constricting member, the constricting member configured to be received over the delivery device with the enlarged head facing toward the tip of the delivery device.

18. The system of claim 17, wherein the enlarged head of the constricting member has an interior diameter sized to receive the locking ring.

19. The system of claim 17, wherein the constricting member is transparent.

20. The system of claim 11, wherein the compression member is transparent.
